(19) United States
(12) Patent Application Publication
(10) Pub. No.: US 2026/0112478 A1
(43) Pub. Date: Apr. 23, 2026

(54) SYSTEMS AND METHODS FOR IDENTIFYING AND LOCATING MEDICAL EQUIPMENT AND/OR SUPPLIES

(71) Applicant: Kimberly Haines, Crystal Lake, IL (US)

(72) Inventor: Kimberly Haines, Crystal Lake, IL (US)

(21) Appl. No.: 19/362,737

(22) Filed: Oct. 20, 2025

Related U.S. Application Data
(60) Provisional application No. 63/709,584, filed on Oct. 21, 2024.

(51) Int. Cl. G16H 40/20; G16H 40/67

(52) U.S. Cl. CPC G16H 40/20; G16H 40/67

(57) ABSTRACT

Disclosed herein are systems and methods for identifying and locating medical equipment and/or supplies. The methods can include transmitting a request for medical equipment and/or supplies, transforming one or more clinical names of the medical equipment and/or supplies to an inventory name, transmitting the inventory name to an inventory database, and identifying at least one location of the medical equipment and/or supplies.

### CROSS-REFERENCE TO RELATED APPLICATIONS

This application claims the benefit of U.S. Application No. 63/709,584, filed Oct. 21, 2024, the entire contents of which are incorporated herein by reference in their entirety.

### Field of Disclosure

The present disclosure relates to systems and methods for identifying and locating medical equipment and/or supplies within a facility. The method can include transmitting a request for medical equipment and/or supplies, transforming a clinical name of the medical equipment and/or supplies to an inventory name of the medical equipment and/or supplies, transmitting the inventory name to an inventory database, identifying a location of the medical equipment and/or supplies, and transmitting the inventory name and location of the medical equipment and/or supplies to a staff computing device.

### BACKGROUND

In recent years, healthcare staff turnover has been high and is continuing to increase. Due to the high rate of healthcare staff turnover, clinical institutional knowledge is declining. Supply chain personnel are not trained clinically or familiar with clinical medical terminology. Often, the clinical medical terminology differs from inventory terminology of medical equipment and supplies. Due to the difference in clinical and inventory terminology, staff responsible for retrieving medical equipment and/or supplies often do not know which medical equipment and/or supplies are needed by the doctor or nurse requesting the equipment and/or supplies. The disconnect in terminology causes nurses and other patient-facing staff to have to go into inventory to retrieve the necessary equipment and/or supplies, thereby wasting valuable time and increasing frustration between clinical and non-clinical staff.

Therefore, there is a need for systems and methods to identify and locate medical equipment and/or supplies, such that inventory staff are able to quickly identify the requested equipment and/or supplies and provide the equipment and/or supplies to the clinical requesting teams.

### SUMMARY

Provided herein is a method for identifying and locating medical equipment and/or supplies. The method can include transmitting, via a clinician computing device connected to at least one server, a request for the medical equipment and/or supplies; transforming, via a name translation model of the at least one server, the one or more clinical names of the medical equipment and/or supplies to an inventory name of the medical equipment and/or supplies; transmitting, via the at least one server, the inventory name of the medical equipment and/or supplies to an inventory database of the at least one server; identifying, at the inventory database, at least one location of the medical equipment and/or supplies; and transmitting, via the at least one server, the inventory name and the at least one location of the medical equipment and/or supplies to a staff computing device.

In some aspects, the inventory name is a unique device identifier. In some aspects, the request is a voice activated request. In some aspects, the method can further include updating the name translation model based on one or more new clinical names for the medical equipment and/or supplies. In some aspects, the one or more clinical name databases include one or more geographical clinical name databases. In some aspects, transforming the one or more clinical names to the inventory name includes accessing one of the one or more geographical clinical name databases based on a geographical location of the request.

In some aspects, the method can further include retrieving the medical equipment and/or supplies and delivering the medical equipment and/or supplies to a clinician in need of the medical equipment and/or supplies. In some aspects, transmitting the at least one location of the medical equipment and/or supplies to the staff computing device includes transmitting a closest location of the medical equipment and/or supplies. In some aspects, the method further includes displaying the inventory name and the closest location of the medical equipment and/or supplies on the staff computing device. In some aspects, the at least one location of the medical equipment and/or supplies includes a plurality of locations of the medical equipment and/or supplies. In some aspects, the method further includes displaying directions to the plurality of locations of the medical equipment and/or supplies on the staff computing device.

Further provided herein is a system for identifying and locating medical equipment and/or supplies. The system can include a clinician computing device, a staff computing device, and at least one processor. The at least one processor can be configured to receive, from the clinician computing device, a request for the medical equipment and/or supplies; transform, via a name translation model, the one or more clinical names of the medical equipment and/or supplies to an inventory name of the medical equipment and/or supplies; and transmit the inventory name and the at least one location of the medical equipment and/or supplies to the staff computing device.

In some aspects, the clinician computing device includes a user input for inputting the request. In some aspects, the user input includes a microphone, a graphical user interface, and/or a keyboard. In some aspects, the at least one processor is configured to update the name translation model based on one or more new clinical names for the medical equipment and/or supplies. In some aspects, the name translation model includes one or more clinical name databases to transform the one or more clinical names to the inventory name. In some aspects, the one or more clinical name databases include one or more geographical name databases.

In some aspects, the staff computing device comprises a display configured to display the inventory name and the at least one location of the medical equipment. In some aspects, the at least one location of the medical equipment and/or supplies includes a plurality of locations of the medical equipment and/or supplies. In some aspects, the staff computing device comprises a display configured to display directions to the plurality of locations.

Other aspects and iterations of the invention are described more thoroughly below.

### BRIEF DESCRIPTION OF FIGURES

The description will be more fully understood with reference to the following figures and graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered limiting of its scope, the principles herein are described and explained with additional specificity and detail through the use of the accompanying drawings in which:

FIG. 1 illustrates a system for identifying and locating medical equipment and/or supplies in one example.

FIG. 2 is a flowchart of a method for identifying and locating medical equipment and/or supplies in one example.

FIG. 3 illustrates an example of a deep learning neural network that can be used to implement a transformation of a clinical name to an inventory name in one example.

FIG. 4 is a diagram illustrating an example of a computing system.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

As used herein, “clinical name” refers to terms used for medical equipment and/or supplies in a clinical setting. Clinical names can be shorthand terms, slang terms, terms used in medical schools, colloquial terms, terms cultivated by regional geography, terms cultivated culturally within an institution, generationally cultivated terms, international terms, or any term commonly understood to refer to certain medical equipment and/or supplies.

As used herein, “inventory name” refers to terms used for medical equipment and/or supplies in an inventory or supply chain setting. Inventory names can include manufacturer names, Food and Drug Administration (FDA) names, unique device identifiers, product names, manufacturing brand names (e.g., Kleenex), global trade item number (GTIN), or any other term commonly understood to refer to certain medical equipment and/or supplies in an inventory or supply chain setting.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

Modern healthcare requires efficient management of thousands of pieces of medical equipment and supplies. Doctors, nurses, and other patient-facing personnel need to have medical equipment supplies delivered to them quickly and efficiently when requested. However, healthcare staff, for example, supply chain staff (e.g., inventory staff), turnover is high and is only growing, making the efficient delivery of medical equipment and supplies increasingly difficult. Compounding the problem of increased turnover is the difference in clinical and inventory naming conventions. For example, many clinicians (e.g., doctors, nurses, and other patient facing personnel) utilize naming conventions that are commonly known to them but are not labeled on the equipment and supplies. Medical equipment and supply clinical names differ tremendously from inventory names, such that many, if not all, items are called entirely different things in the clinical setting than in the inventory/supply chain setting. Recently, the FDA has mandated certain naming conventions for medical equipment and supplies causing increasing frustration in the healthcare industry for tracking pricing, ordering, and invoicing at all points in the supply chain process. Further, the FDA mandated naming conventions do not intuitively connect with clinical language.

Due to the high turnover, institutional knowledge of clinical naming conventions is being lost in inventory staff teams. Increasingly, clinicians must leave the patient to track down inventory staff, or even the medical equipment or supplies themselves, wasting valuable time in treating the current patient or subsequent patients. This loss of efficiency leads to lower quality and/or more time consuming patient care. Further, when clinicians have to leave an operating room, they must scrub out and scrub back in, further exasperating the loss of time in treating the patient. Even further, in emergencies, clinicians need to be able to focus on treating the patient, rather than locating medical equipment and supplies. Therefore, there is a significant need in the medical industry for systems and methods to translate the commonly used clinical names of medical equipment and supplies to inventory names of the same medical equipment and supplies so that inventory staff can efficiently locate the equipment and supplies needed, while the clinicians focus on treating the patient.

Provided herein are systems and methods for identifying and locating medical equipment and supplies. The systems and methods can greatly increase the efficiency of hospital supply management. The systems and methods described herein can quickly and efficiently transform (e.g., translate) clinical names to inventory names, such that inventory staff can easily identify a requested piece of medical equipment or supplies. Further, the systems and methods described herein can be operable to locate the medical equipment and/or supplies such that the inventory staff can be directed to the location efficiently.

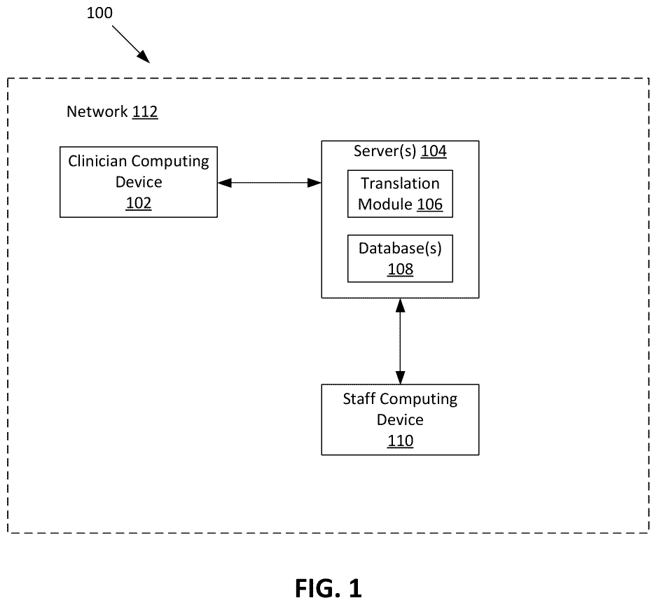

FIG. 1 illustrates a system 100 for identifying and locating medical equipment and supplies in one example. The system can include a clinician computing device 102, a staff computing device 110, and at least one server 104 connected to a network 112. It will be appreciated that clinician computing device 102 can include a plurality of clinician computing devices and that staff computing device 110 can include a plurality of staff computing devices, such that multiple clinicians and multiple staff members can be connected to the network 112.

The clinician computing device 102 can be any computing device configured to transmit requests to the at least one server 104. In some examples, the clinician computing device 102 can be a mobile computing device. For example, the clinician computing device 102 can be a smart phone, smart watch, tablet, or other computing device. In some examples, the clinician computing device 102 can include a user input. In some examples, the user input can include one or more of a microphone, a graphical user interface (e.g., touch screen), and a keyboard. The clinician and/or clinical team member can input a request for medical equipment and/or supplies using one or more clinical names of the medical equipment and/or supplies. For example, the request can include a voice activated request (e.g., via the microphone) or an input request (e.g., via the graphical user interface or keyboard). The clinician computing device 102 can be operable to transmit the request to the at least one server 104 via the network 112. In some examples, the clinician computing device 102 can include a display operable to display an image of the requested medical equipment and/or supplies, such that the clinician and/or clinical team member can ensure that the proper piece of medical equipment and/or supplies is being requested.

The at least one server 104 can be operable to receive the request from the clinician computing device 102. In some examples, the at least one server 104 can include at least one processor. The at least one processor can be configured to analyze the request from the clinician computing device 102. The at least one server 104 can include a name translation module 106 and one or more databases 108. In some examples, the one or more databases 108 can include one or more name databases, one or more inventory databases, and one or more facility map databases.

In some examples, the one or more name databases can include clinical names of medical equipment and supplies (e.g., the term used by clinicians to identify specific pieces of medical equipment or supplies) and inventory names of medical equipment and supplies (e.g., the term used by inventory staff to identify specific pieces of medical equipment or supplies). The one or more name databases can be referred to herein as one or more clinical name databases. The one or more clinical name databases can include both clinical names and corresponding inventory names for medical equipment and/or supplies. For example, the one or more name databases can include both the clinical name for a piece of medical equipment and an inventory name for the piece of medical equipment. In some examples, the one or more name databases can include one or more clinical names for a specific piece of medical equipment that only has a single inventory name. The one or more name databases can be continuously updated with new clinical names for medical equipment. In some examples, the inventory name can include a unique device identifier (UDI).

In some examples, the one or more name databases can be continuously updated with new clinical names for medical equipment and/or supplies. For example, as additional clinical names for medical equipment and/or supplies are learned, the one or more name databases can be updated. The one or more name databases can be updated by receiving additional clinical names and corresponding inventory names. In some examples, when the one or more name databases do not have a match for an input clinical name, the server 104 can transmit a request to the clinician computing device 102 to clarify the clinical name. For example, the clinician computing device 102 can display a message that a clinical name was not found in the one or more databases. A clinician or clinical team member can then input a new clinical name and/or search for the correct piece of medical equipment and/or supplies by inventory name.

In some examples, an inventory name can be a name registered with the FDA by a manufacturer. In some examples, the inventory name can be any name registered with the FDA for a specific piece of medical equipment and/or supplies. For example, an inventory name for a specific piece of medical equipment can be neuro sponges and the clinical name for the same piece of medical equipment can be neuro patties and/or cottonoid. In another example, an inventory name for a specific piece of medical equipment can be electrosurgery device and the clinical name can be cautery, Bovie, and/or diathermy pencil. The one or more name databases can be configured to translate the clinical name to the inventory name.

In some examples, the one or more name databases can include one or more geographical name databases which can include data related to clinical naming conventions in various geographical regions. For example, a first clinician may use a first term to identify a specific piece of medical equipment and/or supplies in a first geographical region and a second clinician may use a second term to identify the same specific piece of medical equipment and/or supplies in a second geographical region. Each geographical name database can include data for a specific region such that the term used for a specific piece of medical equipment and/or supplies in that geographical region is stored in the geographical database. For example, in a first geographical region, a clinical team member can request a neuro patty which has an inventory name of neuro sponge. In a second geographical region, a clinical team member can request a cottonoid which has the same inventory name of neuro sponge. Based on the geographical location of the clinical team member, the one or more geographical name databases can be configured to translate the clinical name for the requested medical equipment to the inventory name. In another example, in a first geographical region (e.g., western United States), a clinical team member can request a cautery which has an inventory name of electrosurgery device. In a second geographical region (e.g., eastern United States), a clinical team member can request a Bovie which has an inventory name of electrosurgery device. In a third geographical region (e.g., England), a clinical team member can request a diathermy pencil which has an inventory name of electrosurgery device. Based on the geographical location of the clinical team member, the one or more geographical name databases can be configured to translate the clinical name for the requested medical equipment and/or supplies to the inventory name.

The one or more inventory databases can include a location and a quantity of the medical equipment and supplies. The one or more inventory databases can include a specific location in a facility where specific pieces of medical equipment and supplies are located. For example, the location can include a specific floor and/or room. The one or more inventory databases can be continuously updated as medical equipment and supplies are used and replenished. Inventory staff can scan or otherwise track use of specific pieces of medical equipment and supplies, such that the one or more inventory databases remain continuously updated.

In some examples, the one or more facility map databases can include floorplans of facilities. For example, the one or more facility map databases can include the location of a room based on floor and room number. The one or more facility map databases can include data collected from specific facilities (e.g., hospitals, outpatient centers, and other medical facilities).

The name translation module 106 can be in communication with the one or more databases 108. For example, when a request for medical equipment and/or supplies is obtained from the clinician computing device 102, the name translation module 106 can access the one or more name databases and transform (e.g., translate) the clinical name used in the request to an inventory name. In some examples, the at least one server 104 can then transmit the inventory name to the staff computing device 110. In this manner, the inventory staff can quickly determine the pieces of medical equipment and/or supplies requested, without the need for a clinician to provide further explanation or translation.

In some examples, the name translation module 106 can be a machine learning module, an artificial intelligence module, a data analysis module, or another type of computer program configured to transform the clinical name in the request to an inventory name. For example, the name translation module 106 can be configured to receive the clinical name used in the request for the medical equipment and/or supplies. The name translation module 106 can access the one or more name databases. The name translation module 106 can then use the clinical name in the request from the clinician computing device 102 and transform the clinical name into the inventory name of the medical equipment and/or supplies. In some examples, the name translation module 106 can receive a geographical location of the clinician computing device 102 and transform the clinical name to the inventory name using the one or more geographical name databases based on the geographical location of the clinician computing device 102. The at least one server 104 can then transmit the inventory name of the medical equipment and/or supplies to the staff computing device 110.

In some examples, the at least one server 104 can transmit a location of the medical equipment and/or supplies to the staff computing device 110 along with the inventory name. For example, the at least one processor of the at least one server 104 can access the one or more inventory databases and determine a location of the requested medical equipment and/or supplies. In conjunction with the inventory name, the location of the requested medical equipment and/or supplies can allow for inventory staff to locate the requested medical equipment and/or supplies quickly and efficiently. In some examples, the location of the medical equipment and/or supplies can include a plurality of locations of the medical equipment and/or supplies within the facility. When the requested medical equipment and/or supplies has a plurality of locations, the at least one processor can access the location of the staff computing device 110 and the one or more facility map databases to determine a closest location of the requested medical equipment and/or supplies. For example, the at least one processor can determine the closest location of the requested medical equipment and/or supplies to the staff computing device 110. The at least one processor can transmit the closest location of the requested medical equipment and/or supplies to the staff computing device 110. In some examples, the closest location of the requested medical equipment and/or supplies can be displayed on the staff computing device 110.

In some examples, the at least one server 104 can also transmit directions to the location of the medical equipment and/or supplies to the staff computing device 110 along with the inventory name. For example, the at least one processor of the at least one server 104 can access the one or more facility databases. The at least one processor of the at least one server 104 can also receive a location of the staff computing device 110. The at least one processor of the at least one server 104 can then provide directions from the staff computing device 110 to the location of the medical equipment and/or supplies utilizing a facility map from the one or more facility databases. In some examples, the at least one processor of the at least one server 104 can further transmit directions from the staff computing device 110 to the clinician utilizing locations of the clinician computing device 102, the staff computing device 110, and a facility map from the one or more facility map databases.

In some examples, the staff computing device 110 can include a display. The display can be configured to display the inventory name of the requested medical equipment and/or supplies, the location of the requested medical equipment and/or supplies, directions to the requested medical equipment and/or supplies, and/or directions to the clinician requesting the medical equipment and/or supplies. In this manner, the inventory staff can easily locate the requested medical equipment and/or supplies and easily locate the clinician requesting the medical equipment and/or supplies.

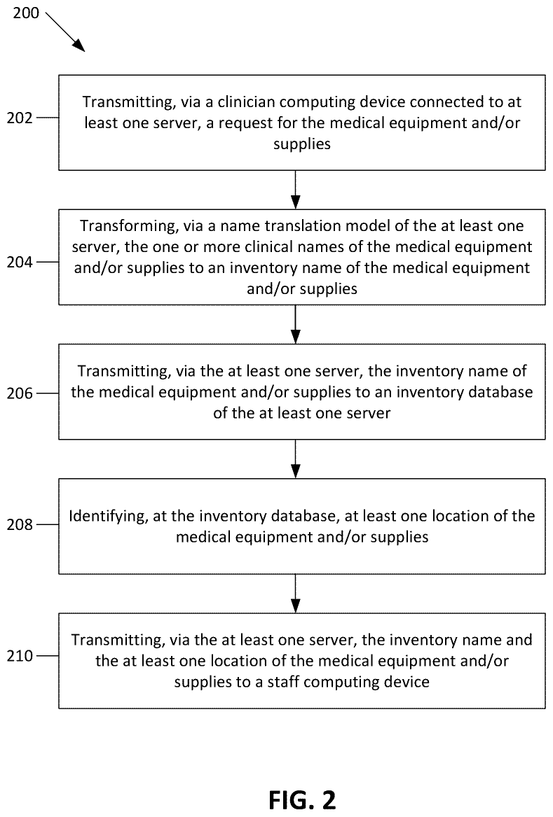

FIG. 2 illustrates an example method 200 for identifying and locating medical equipment and/or supplies. In some examples, the method 200 can utilize one or more components of the system described herein.

At step 202, the method 200 can include transmitting, via a clinician computing device connected to at least one server, a request for the medical equipment and/or supplies. In some examples, the request for the medical equipment and/or supplies can include one or more clinical names of the medical equipment and/or supplies. In some examples, the one or more clinical names can be different than an inventory name of the medical equipment and/or supplies. In some examples, the one or more clinical names can differ depending on a geographical location of the clinician and/or depending on the specific clinician making the request.

In some examples, the request for the medical equipment and/or supplies can be a voice activated request (e.g., via a microphone of the clinician computing device). In some examples, the request can be input into the clinician computing device via a graphical user interface (e.g., touch screen) or a keyboard. In some examples, the clinician computing device is a mobile computing device.

At step 204, the method 200 can include transforming, via a name translation model of the at least one server, the one or more clinical names of the medical equipment and/or supplies to an inventory name of the medical equipment and/or supplies. In some examples, the inventory name can be a unique device identifier (UDI). In some examples, the name translation model can access one or more databases when transforming the one or more clinical names to an inventory name. For example, the name translation model can access one or more name databases that include one or more clinical names for specific pieces of medical equipment and/or supplies and corresponding inventory names of the medical equipment and/or supplies. In some examples, the name translation module can be a machine learning model, an artificial intelligence model, a data analysis model, or other computer programs operable to transform (e.g., translate) one or more clinical names of a piece of medical equipment and/or supplies into an inventory name of the piece of medical equipment and/or supplies.

In some examples, the one or more name databases can include one or more geographical name databases. For example, different geographical regions can have different clinical names for a specific piece of medical equipment. In some examples, the name translation model and/or the at least one server can receive geographical location data from the clinician computing device. The name translation model can then access the correct geographical name database based on the location of the clinician computing device, such that the clinical name is transformed to the correct inventory name. In this manner, the name translation model can ensure that the desired medical equipment and/or supplies is identified by the inventory name.

In some examples, the one or more name databases can be continuously updated with new clinical names, new inventory names, and new medical equipment and supplies.

At step 206, the method 200 can include transmitting, via the at least one server, the inventory name of the medical equipment and/or supplies to an inventory database of the at least one server.

At step 208, the method 200 can include identifying, at the inventory database, at least one location of the medical equipment and/or supplies. In some examples, the inventory database can include a plurality of locations of the medical equipment and/or supplies. In some examples, the inventory database is continuously updated as medical equipment and supplies are used or replenished. In some examples, the medical equipment and/or supplies can be scanned or otherwise tracked to the location the medical equipment and/or supplies are located. In this manner, the inventory database includes updated data for the location and/or quantity of the medical equipment and/or supplies in the facility.

At step 210, the method 200 can include transmitting, via the at least one server, the inventory name and the at least one location of the medical equipment and/or supplies to a staff computing device. In some examples, the method 200 can further include displaying the inventory name and the at least one location of the medical equipment and/or supplies on a display of the staff computing device. In some examples, the clinician computing device can display an image of the requested medical equipment and/or supplies before the inventory name is transmitted to the staff computing device, such that the clinician can confirm that the correct piece of medical equipment and/or supplies is transmitted to the staff computing device.

In some examples, the at least one location of the medical equipment and/or supplies can include a plurality of locations of the medical equipment and/or supplies. For example, the facility can include multiple storage areas where the medical equipment and/or supplies are located. In some examples, the method 200 can include determining a closest location of the medical equipment and/or supplies to the staff computing device. In some examples, the closest location of the medical equipment and/or supplies can be displayed on the staff computing device.

In some examples, the at least one server can include one or more facility map databases. In some examples, at least one processor of the at least one server can be operable to determine directions to the medical equipment and/or supplies based on a location of the staff computing device and the at least one location of the medical equipment and/or supplies. In some examples, the at least one processor can transmit the directions to the staff computing device. In some examples, the staff computing device can be configured to display the directions to the medical equipment and/or supplies.

In some examples, the at least one processor can also determine directions to the clinician in need of the medical equipment and/or supplies based on the one or more facility map databases. In some examples, the at least one processor of the at least one server can transmit the directions to the clinician in need of the medical equipment and/or supplies to the staff computing device. In some examples, the staff computing device can display the directions to the clinician in need of the medical equipment and/or supplies.

In some examples, the method 200 can further include retrieving the medical equipment and/or supplies. In some examples, the method 200 can further include delivering the medical equipment and/or supplies to the clinician in need of the medical equipment and/or supplies. In some examples, the method 200 can include performing an operation (e.g., surgical operation, etc.) with the medical equipment and/or supplies.

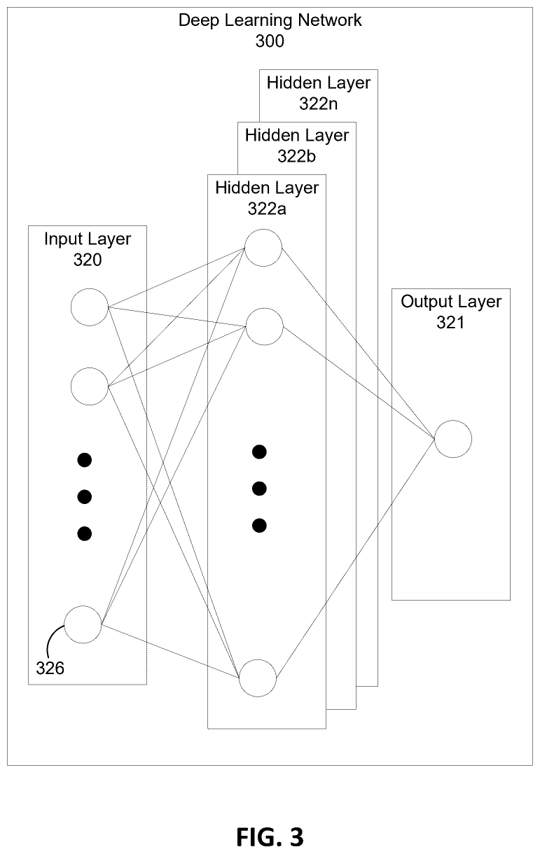

Various aspects of the present disclosure can use machine learning models or systems. FIG. 3 is an illustrative example of a deep learning neural network 300 that can be used to implement the machine learning-based name translation described herein. An input layer 320 includes input data. In one illustrative example, the input layer 320 can include data representing clinical names of the medical equipment and/or medical supplies. The neural network 300 includes multiple hidden layers 322a, 322b, through 322n. The hidden layers 322a, 322b, through 322n include “n” number of hidden layers, where “n” is an integer greater than or equal to one. The number of hidden layers can be made to include as many layers as needed for the given application. The neural network 300 further includes an output layer 321 that provides an output resulting from the processing performed by the hidden layers 322a, 322b, through 322n. In one illustrative example, the output layer 321 can provide a classification for an inventory name of the medical equipment and/or supplies. The classification can include a class identifying the type of activity or object (e.g., inventory name, etc.).

The neural network 300 is a multi-layer neural network of interconnected nodes. Each node can represent a piece of information. Information associated with the nodes is shared among the different layers and each layer retains information as information is processed. In some cases, the neural network 300 can include a feed-forward network, in which case there are no feedback connections where outputs of the network are fed back into itself. In some cases, the neural network 300 can include a recurrent neural network, which can have loops that allow information to be carried across nodes while reading in input.

Information can be exchanged between nodes through node-to-node interconnections between the various layers. Nodes of the input layer 320 can activate a set of nodes in the first hidden layer 322a. For example, as shown, each of the input nodes of the input layer 320 is connected to each of the nodes of the first hidden layer 322a. The nodes of the first hidden layer 322a can transform the information of each input node by applying activation functions to the input node information. The information derived from the transformation can then be passed to and can activate the nodes of the next hidden layer 322b, which can perform their own designated functions. Example functions include convolutional, up-sampling, data transformation, and/or any other suitable functions. The output of the hidden layer 322b can then activate nodes of the next hidden layer, and so on. The output of the last hidden layer 322n can activate one or more nodes of the output layer 321, at which an output is provided. In some cases, while nodes (e.g., node 326) in the neural network 300 are shown as having multiple output lines, a node has a single output and all lines shown as being output from a node represent the same output value.

In some cases, each node or interconnection between nodes can have a weight that is a set of parameters derived from the training of the neural network 300. Once the neural network 300 is trained, it can be referred to as a trained neural network, which can be used to classify one or more activities. For example, an interconnection between nodes can represent a piece of information learned about the interconnected nodes. The interconnection can have a tunable numeric weight that can be tuned (e.g., based on a training dataset), allowing the neural network 300 to be adaptive to inputs and able to learn as more and more data is processed.

The neural network 300 is pre-trained to process the features from the data in the input layer 320 using the different hidden layers 322a, 322b, through 322n in order to provide the output through the output layer 321. In an example in which the neural network 300 is used to identify inventory names, the neural network 300 can be trained using training data that includes both clinical names and inventory names, as described herein. For instance, training data including clinical names and inventory names of medical equipment and/or supplies can be input into the network, with each training frame having a label indicating the inventory name (for a feature extraction machine learning model).

The neural network 300 can include any suitable deep network. One example includes a convolutional neural network (CNN), which includes an input layer and an output layer, with multiple hidden layers between the input and out layers. The hidden layers of a CNN include a series of convolutional, nonlinear, pooling (for downsampling), and fully connected layers. The neural network 300 can include any other deep network other than a CNN, such as an autoencoder, a deep belief nets (DBNs), a Recurrent Neural Networks (RNNs), BNNs, among others.

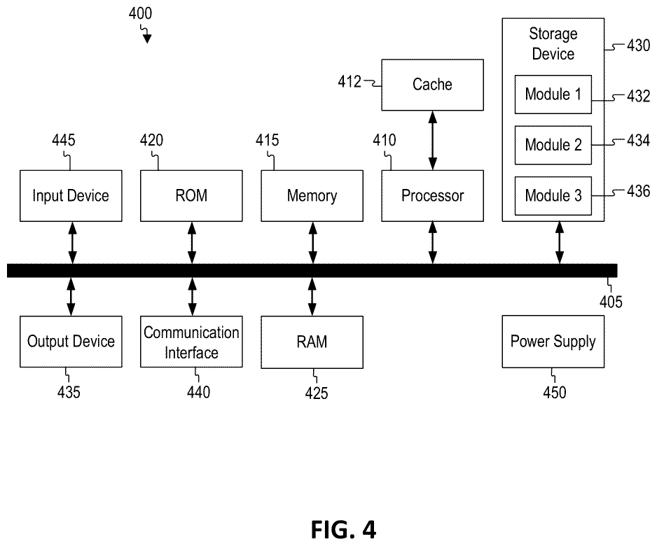

FIG. 4 shows an example of computing system 400, which can be for example any computing device making up various parts of the medical equipment and/or supplies identification and location system (e.g., at least one server, clinician computing device, and/or staff computing device), or any component thereof in which the components of the system are in communication with each other using connection 405. Connection 405 can be a physical connection via a bus, or a direct connection into processor 410, such as in a chipset architecture. Connection 405 can also be a virtual connection, networked connection, edge network connection, or logical connection.

In some embodiments, computing system 400 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example system 400 includes at least one processing unit (CPU or processor) 410 and connection 405 that couples various system components including system memory 415, such as read-only memory (ROM) 420 and random-access memory (RAM) 425 to processor 410. Computing system 400 can include a cache of high-speed memory 412 connected directly with, in close proximity to, or integrated as part of processor 410.

Processor 410 can include any general purpose processor and a hardware service or software service, such as services 432, 432, and 436 stored in storage device 430, configured to control processor 410 as well as a special-purpose processor where software instructions are incorporated into the actual processor design. Processor 410 may essentially be a completely self-contained computing system, containing multiple cores or processors, a bus, memory controller, cache, etc. A multi-core processor may be symmetric or asymmetric.

To enable user interaction, computing system 400 includes an input device 425, which can represent any number of input mechanisms, such as a microphone for speech, a touch-sensitive screen for gesture or graphical input, keyboard, mouse, motion input, speech, etc. Computing system 400 can also include output device 435, which can be one or more of a number of output mechanisms known to those of skill in the art. In some instances, multimodal systems can enable a user to provide multiple types of input/output to communicate with computing system 400. Computing system 400 can include communications interface 420, which can generally govern and manage the user input and system output. There is no restriction on operating on any particular hardware arrangement, and therefore the basic features here may easily be substituted for improved hardware or firmware arrangements as they are developed.

Storage device 430 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 430 can include software services, servers, services, etc., that when the code that defines such software is executed by the processor 410, it causes the system to perform a function. In some embodiments, a hardware service that performs a particular function can include the software component stored in a computer-readable medium in connection with the necessary hardware components, such as processor 410, connection 405, output device 435, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

### Exemplary Clauses

Clause 1. A method for identifying and locating medical equipment and/or supplies, the method comprising: transmitting, via a clinician computing device connected to at least one server, a request for the medical equipment and/or supplies, wherein the request for the medical equipment and/or supplies includes one or more clinical names of the medical equipment; transforming, via a name translation model of the at least one server, the one or more clinical names of the medical equipment and/or supplies to an inventory name of the medical equipment and/or supplies; transmitting, via the at least one server, the inventory name of the medical equipment and/or supplies to an inventory database of the at least one server; identifying, at the inventory database, at least one location of the medical equipment and/or supplies; and transmitting, via the at least one server, the inventory name and the at least one location of the medical equipment and/or supplies to a staff computing device.

Clause 2. The method of clause 1, wherein the inventory name is a unique device identifier.

Clause 3. The method of clause 1, wherein the request is a voice activated request.

Clause 4. The method of clause 1, the method further comprising updating the name translation model based on one or more new clinical names for the medical equipment and/or supplies.

Clause 5. The method of clause 1, wherein the name translation model accesses one or more clinical name databases to transform the one or more clinical names to the inventory name.

Clause 6. The method of clause 5, wherein the one or more clinical name databases include one or more geographical clinical name databases.

Clause 7. The method of clause 6, wherein transforming the one or more clinical names to the inventory name comprises accessing one of the one or more geographical clinical name databases based on a geographical location of the request.

Clause 8. The method of clause 1, the method further comprising: retrieving the medical equipment and/or supplies; and delivering the medical equipment and/or supplies to a clinician in need of the medical equipment and/or supplies.

Clause 9. The method of clause 1, wherein transmitting the at least one location of the medical equipment to the staff computing device includes transmitting a closest location of the medical equipment and/or supplies.

Clause 10. The method of clause 9, the method further comprising displaying the inventory name and the closest location of the medical equipment and/or supplies on the staff computing device.

Clause 11. The method of clause 1, wherein the at least one location of the medical equipment and/or supplies includes a plurality of locations of the medical equipment and/or supplies.

Clause 12. The method of clause 11, the method further comprising displaying directions to the plurality of locations of the medical equipment and/or supplies on the staff computing device.

Clause 13. A system for identifying and locating medical equipment and/or supplies, the system comprising: a clinician computing device; a staff computing device; and at least one processor, the at least one processor operable to: receive, from the clinician computing device, a request for the medical equipment and/or supplies, wherein the request for the medical equipment and/or supplies includes one or more clinical names of the medical equipment and/or supplies; transform, via a name translation model, the one or more clinical names of the medical equipment and/or supplies to an inventory name of the medical equipment and/or supplies; identify, via an inventory database, at least one location of the medical equipment and/or supplies; and transmit the inventory name and the at least one location of the medical equipment and/or supplies to the staff computing device.

Clause 14. The system of clause 13, wherein the clinician computing device includes a user input for inputting the request, wherein the user input includes a microphone, graphical user interface, and/or keyboard.

Clause 15. The system of clause 13, wherein the at least one processor is configured to update the name translation model based on one or more new clinical names for the medical equipment and/or supplies.

Clause 16. The system of clause 13, wherein the name translation model includes one or more clinical name databases to transform the one or more clinical names to the inventory name.

Clause 17. The system of clause 16, wherein the one or more clinical name databases include one or more geographical clinical name databases.

Clause 18. The system of clause 13, wherein the staff computing device comprises a display configured to display the inventory name and the at least one location of the medical equipment and/or supplies.

Clause 19. The system of clause 13, wherein the at least one location of the medical equipment and/or supplies includes a plurality of locations of the medical equipment and/or supplies.

Clause 20. The system of clause 19, wherein the staff computing device comprises a display configured to display directions to the plurality of locations.

What is claimed is:

1. A method for identifying and locating medical equipment and/or supplies, the method comprising:
transmitting, via a clinician computing device connected to at least one server, a request for the medical equipment and/or supplies, wherein the request for the medical equipment and/or supplies includes one or more clinical names of the medical equipment;
transforming, via a name translation model of the at least one server, the one or more clinical names of the medical equipment and/or supplies to an inventory name of the medical equipment and/or supplies;
transmitting, via the at least one server, the inventory name of the medical equipment and/or supplies to an inventory database of the at least one server;
identifying, at the inventory database, at least one location of the medical equipment and/or supplies; and
transmitting, via the at least one server, the inventory name and the at least one location of the medical equipment and/or supplies to a staff computing device.

2. The method of claim 1, wherein the inventory name is a unique device identifier.

3. The method of claim 1, wherein the request is a voice activated request.

4. The method of claim 1, the method further comprising updating the name translation model based on one or more new clinical names for the medical equipment and/or supplies.

5. The method of claim 1, wherein the name translation model accesses one or more clinical name databases to transform the one or more clinical names to the inventory name.

6. The method of claim 5, wherein the one or more clinical name databases include one or more geographical clinical name databases.

7. The method of claim 6, wherein transforming the one or more clinical names to the inventory name comprises accessing one of the one or more geographical clinical name databases based on a geographical location of the request.

8. The method of claim 1, the method further comprising:
delivering the medical equipment and/or supplies to a clinician in need of the medical equipment and/or supplies.

9. The method of claim 1, wherein transmitting the at least one location of the medical equipment to the staff computing device includes transmitting a closest location of the medical equipment and/or supplies.

10. The method of claim 9, the method further comprising displaying the inventory name and the closest location of the medical equipment and/or supplies on the staff computing device.

11. The method of claim 1, wherein the at least one location of the medical equipment and/or supplies includes a plurality of locations of the medical equipment and/or supplies.

12. The method of claim 11, the method further comprising displaying directions to the plurality of locations of the medical equipment and/or supplies on the staff computing device.

13. A system for identifying and locating medical equipment and/or supplies, the system comprising:
a clinician computing device;
a staff computing device; and
at least one processor, the at least one processor operable to:
receive, from the clinician computing device, a request for the medical equipment and/or supplies, wherein the request for the medical equipment and/or supplies includes one or more clinical names of the medical equipment and/or supplies;
transform, via a name translation model, the one or more clinical names of the medical equipment and/or supplies to an inventory name of the medical equipment and/or supplies;
identify, via an inventory database, at least one location of the medical equipment and/or supplies; and
transmit the inventory name and the at least one location of the medical equipment and/or supplies to the staff computing device.

14. The system of claim 13, wherein the clinician computing device includes a user input for inputting the request, wherein the user input includes a microphone, graphical user interface, and/or keyboard.

15. The system of claim 13, wherein the at least one processor is configured to update the name translation model based on one or more new clinical names for the medical equipment and/or supplies.

16. The system of claim 13, wherein the name translation model includes one or more clinical name databases to transform the one or more clinical names to the inventory name.

17. The system of claim 16, wherein the one or more clinical name databases include one or more geographical clinical name databases.

18. The system of claim 13, wherein the staff computing device comprises a display configured to display the inventory name and the at least one location of the medical equipment and/or supplies.

19. The system of claim 13, wherein the at least one location of the medical equipment and/or supplies includes a plurality of locations of the medical equipment and/or supplies.

20. The system of claim 19, wherein the staff computing device comprises a display configured to display directions to the plurality of locations.
